(19) United States
(12) Patent Application Publication
(10) Pub. No.: US 2011/0160137 A1
(43) Pub. Date: Jun. 30, 2011

(54) COMPOSITION CONTAINING COLLAGEN PEPTIDE FOR IMPROVING SKIN CARE

(75) Inventors: Jeong Kee Kim, Yongin-si (KR); Ji Hae Lee, Gyeonggi-do (KR); Mi Suk Yang, Gyeonggi-do (KR); Ji Eun Lee, Gyeonggi-do (KR); Wan Gi Kim, Gyeonggi-do (KR)

(73) Assignee: AMOREPACIFIC CORPORATION, Seoul (KR)

(21) Appl. No.: 13/060,825

(22) PCT Filed: Aug. 27, 2009
(86) PCT No.: PCT/KR2009/004793

§ 371 Date: Feb. 25, 2011

(30) Foreign Application Priority Data
Aug. 27, 2008 (KR) 10-2008-0083759

(51) Int. Cl. A61K 8/65; A61Q 19/08; A61Q 19/00

(52) U.S. Cl. 514/17.2

(57) ABSTRACT

The present invention relates to an oral composition for improving the beauty of the skin, which exhibits the effects of reducing skin wrinkles and inhibiting wrinkle formation. The composition contains a collagen peptide and at least one selected from the group consisting of elastin protein, hyaluronic acid and vitamin C. Particularly, the composition contains the collagen peptide, the elastin protein, hyaluronic acid and vitamin C at the optimum ratio, and when it is taken into the human body, it has no side effect, maximizes the biosynthesis of collagen in the skin dermal layer, shows excellent in vivo retention rate, and exhibits the effects of inhibiting skin wrinkle formation, maintaining or improving skin elasticity and moisturizing the skin. Thus, the composition will be useful as a health functional food for improving the beauty of the skin and preventing skin aging.

### TECHNICAL FIELD

The present invention relates to a composition for improving the beauty of the skin, which contains a collagen peptide mixture, and more particularly to a composition for improving the beauty of the skin, which contains a collagen peptide and at least one selected from the group consisting of hyaluronic acid, elastin protein and vitamin C, and thus has an excellent effect of improving skin conditions.

### BACKGROUND ART

Skin aging can be classified into intrinsic aging and extrinsic aging depending on its cause. Intrinsic aging is a process by which the skin structure and the physiological functions of the skin deteriorate regardless of environmental changes as people grow older. Extrinsic aging is caused by continuous exposure to external environment such as sunlight. Especially, skin aging caused by light is called photoaging. Ultraviolet (UV) light is the main cause of physiological and morphological changes of the skin aging. As intrinsic skin aging proceeds, the skin becomes dry, while fine wrinkles increase and deepen. Further, because of structural and functional changes of the epidermis, the dermis, and the like, the skin loses much of its elasticity and looks drooping. The dermis becomes thinner, whereas the total quantity of collagen is lost 1% each year for adults. Also, the remaining collagen fibers gradually become thicker, while the cross-linking thereof increases, so that the solubility, elasticity and like thereof decrease. In addition, elastin fibers become thicker and the cross-linking thereof also increase. Moreover, the proliferative activity of fibroblasts in the dermis decreases, and the ability to synthesize and degrade collagen also decreases. Collagen is the main component of skin tissue associated with skin aging. The collagen protein accounts for 77% of the total dry weight of the skin, excluding fats, and accounts for 90% of the fibrous components of the dermis. It is responsible for maintaining skin strength, elasticity and flexibility. Accordingly, the promotion of collagen synthesis and the inhibition of collagen degradation have become a major issue with regard to skin beauty and prevention of skin aging, and it is required to develop beauty foods that can inhibit photoaging caused by UV light while promoting collagen synthesis.

For this purpose, studies on collagen products that can be orally taken have been continuously conducted. With the development of technology, collagen products from various sources and collagen materials that underwent various post-treatment processes have been developed. In connection with this, Korean Patent Laid-Open Publication No. 2001-0075842 (Aug. 11, 2001) discloses a functional food containing collagen, and Korean Patent Laid-Open Publication No. 2002-0085307 (Nov. 16, 2002) discloses an oral food composition for skin beauty containing a large amount of collagen. However, because collagen has high molecular weight, there have been various questions about the digestion and absorption of collagen, the delivery of collagen to target organs such as skin, the delivery of an effective amount of collagen to target organs, the biocompatibility of collagen, and the like. Thus, additional studies on the substantial effects of collagen-based materials have been required.

### DISCLOSURE OF INVENTION

Accordingly, the present inventors have conducted extensive studies to find collagen-based formulations exhibiting the effect of improving the beauty of the skin and, as a result, have found that a collagen peptide containing a high concentration of tripeptide in the form of Gly-X-Y has the effect of improving the beauty of the skin. Based on this finding, the present inventors have conducted studies on components promoting the biosynthesis of procollagen in skin cells and, as a result, have found that, if elastin protein, hyaluronic acid and vitamin C are added to the collagen peptide in the optimum amounts, the effect of improving the beauty of the skin can be maximized, thereby completing the present invention.

It is, therefore, an object of the present invention to provide a composition for improving the beauty of the skin, which has no side effects, prevents skin aging by promoting procollagen biosynthesis and inhibiting photoaging and exhibits an excellent effect of improving skin conditions by increasing skin elasticity.

To achieve the above object, the present invention provides a composition for improving the beauty of the skin, which contains a collagen peptide and at least one selected from the group consisting of elastin protein, hyaluronic acid and vitamin C.

The collagen peptide preferably contains a collagen tripeptide in an amount of 15 wt % or more based on the total weight of the collagen peptide.

### EFFECTS OF THE INVENTION

The composition for improving the beauty of the skin according to the present invention can prevent skin aging by promoting collagen synthesis and inhibiting photoaging and also exhibit an excellent effect of improving skin conditions by promoting wound healing and increasing the in vivo retention rate of the collagen peptide.

### BRIEF DESCRIPTION OF THE DRAWINGS

FIG. 1 shows deriving the optimum conditions using a response optimization tool,

FIG. 2 is a contour diagram showing the results of carrying out a central composite design.

FIG. 3 is a set of photographs showing a comparison between skin replicas sampled from animals of Comparative Examples 2 and 3 and Example 1.

FIG. 4 shows the results of immunohistochemical staining conducted to examine the expression of collagen in skin tissue.

FIG. 5 shows the results of analyzing the in vivo retention rates of Comparative Examples 1 and 2 and Example 1.

### BEST MODE FOR CARRYING OUT THE INVENTION

The composition for improving the beauty of the skin according to the present invention contains a collagen peptide and at least one selected from the group consisting of elastin protein, hyaluronic acid and vitamin C.

When analyzing the functions of the above components, an integrated experimental and analytical process is required to obtain the maximum information within given cost and time estimates. For this purpose, in the present invention, DOE (Design of Experiments) was used. The DOE is a methodology for systematically designing, performing and statistically analyzing a science study. Particularly, according to the DOE, a series of experimental steps are designed and carried out, wherein the levels of a controllable input value are gradually varied in order to determine the cause of variance in an output value of a specific step. By doing so, it is possible to construct an experimental strategy for determining experimental conditions under which the maximum information can be obtained efficiently so as to satisfy the objects of the experiment and to obtain adequate results. The DOE allows systematic approach to calibration and allows definition and evaluation of relationships between an experimental process and the resultant values thereof. Additionally, the DOE enables understanding of the vital few in variance of a parameter, provides a measurement for the effect of the vital few upon response parameters, yields effective measurement values and high-quality data as compared to One-Factor-At-a-Time calibration, permits measurement of uncertainty, minimizes test trials, and permits control of the nuisance variables. Such DOEs include fractional factorial designs, full factorial designs, response surface methodology, mixture designs, Taguchi designs, and the like.

The collagen peptide that is used as an active ingredient in the present invention has a molecular weight of 500-1,000 Da and contains a tripeptide in the form of Gly-X-Y in an amount of 15 wt % or more, for example, 15-95%, based on the total weight of the collagen peptide where each of X and Y may be any amino acid. In addition, X and Y may be the same or different amino acids and may also be selected from among all possible combinations of amino acids. Namely, X and Y may be selected from among naturally occurring amino acids, including alanine (Ala), valine (Val), leucine (Leu), isoleucine (Ile), proline (Pro), hydroxyproline (Hyp), phenylalanine (Phe), tryptophan (Trp), methionine (Met), serine (Ser), threonine (Thr), cysteine (Cys), glutamine (Gln), glycine (Gly), asparagines (Asn), tyrosine (Tyr), lysine (Lys), arginine (Arg), histidine (His), aspartic acid (Asp) and glutamic acid (Glu). Preferably, the tripeptide that is used in the present invention may be selected from the group consisting of, but not limited to, Gly-Pro-Hyp, Gly-Pro-Ala, Aly-Ala-Hyp, Gly-Leu-Hyp, Gly-Glu-Lys, Gly-Pro-Lys, Gly-Glu-Hyp, Gly-Phe-Hyp, Gly-Ser-Hyp, Gly-Gln-Hyp, Gly-Glu-Arg, and Gly-Pro-Arg.

Specifically, the collagen peptide that is used in the present invention may be prepared in the following manner. When a collagen or gelatin component is degraded using an enzyme, such as cysteine protease, pepsin, trypsin or collagenase, a collagen peptide containing 5 wt % or more of a tripeptide and a dipeptide can be prepared. In order to increase the tripeptide content of the peptide mixture thus prepared, the peptide mixture containing the tripeptide in the form of Gly-X-Y is brought into contact with alkaline anion exchange resin, the tripeptide Gly-X-Y is adsorbed onto the ion exchange resin, and then the tripeptide is eluted from the ion exchange resin adsorbed with the tripeptide, thereby preparing a purified peptide having a higher tripeptide content. Alternatively, a purified peptide having a higher hydrophilic tripeptide content can be prepared by bringing the peptide mixture containing the tripeptide Gly-X-Y into contact with a nonpolar adsorbent, adsorbing a portion of the hydrophobic peptide contained in the peptide mixture onto the nonpolar adsorbent, and recovering the portion of the hydrophobic tripeptide that was not adsorbed onto the nonpolar adsorbent.

In the present invention, the collagen peptide is contained in an amount of 1-80 wt % based on the total weight of the composition. If the content of the collagen peptide in the composition is less than 1 wt %, it will be difficult to obtain the desired effect, and if the content is more than 80 wt %, it will be difficult to formulate the composition.

Also, the elastin protein that is used in the present invention is an elastin obtained from the aortic bulb of Euthynus pelamis by hydrolysis and is characterized in that it contains desmosine and isodesmosine which are the characteristic crosslinked amino acids of elastin.

The hyaluronic acid that is used in the present invention is produced in large amounts by fermentation of Streptococcus so as to have a hyaluronic acid content of 90-100%. It is widely used in the cosmetic and pharmaceutical fields and has recently been used in various applications, including health supplement foods.

The collagen peptide, elastin protein, hyaluronic acid and vitamin C in the composition of the present invention are contained in the amounts of 1-80 wt %, 1-20 wt %, 1-10 wt % and 1-20 wt %, respectively, based on the total weight of the composition.

The composition according to the present invention can promote the synthesis of collagen in the skin, inhibit the photoaging of the skin, increase skin moisturization, promote wound healing and increase the in vivo retention rate of the collagen peptide, thereby improving skin conditions.

Also, the content ratio of collagen peptide:elastin protein:vitamin C:hyaluronic acid is preferably 1:0.0001-150:0.0001-20:0.0001-50000.

The inventive composition for improving the beauty of the skin may be formulated in various forms, including pills, tea bags, instant teas, drinks, granules, tablets and capsules, which can be used in various applications, including health foods and pharmaceutical drugs.

### MODE FOR INVENTION

Hereinafter, the present invention will be described in further detail by way of examples and test examples with reference to the accompanying drawings. Theses examples and test examples are merely intended to facilitate the understanding of the present invention, and the scope of the present invention is not limited thereto. It will be understood by those skilled in the art that variations, substitutions and insertions which are commonly well-known can be made to the present invention without departing from the spirit and scope of the appended claims.

### Experimental Example 1

### Measurement of the Ability to Produce Collagen

The ability to produce type 1 procollagen was measured using fibroblasts as shown in Table 1 below.

TABLE 1

| Classification | Treatment |
|---|---|
| Control group | In a normal group, cells were treated with |
| | physiological saline. |
| Comparative | Cells are treated with general collagen |
| Example 1 | material dissolved in physiological saline. |
| Comparative | Cells were treated with collagen peptide |
| Example 2 | (purchased from Jellice Co., Ltd.) dissolved |
| | in physiological saline. |

To compare the ability to produce procollagen between the groups, fibroblasts were added to a 48-well plate at a density of 5×10⁴ cells/well and allowed to adhere to each well. In this state, each of the collagens of Comparative Examples 1 and 2 was added to the cells to a final concentration of each of 0.1 ppm, 1 ppm and 10 ppm. As a control group, a group not treated with any collagen sample was prepared. After the cells have been cultured for 48 hours, the production of type 1 procollagen in the supernatant of the culture was measured using an ELISA kit (Takara MK101). The results of the measurement are shown in Table 2 below and expressed relative to the control group taken as 100%.

TABLE 2
Production of Procollagen

| | Concentration | Production of |
|---|---|---|
| Classification | (ppm) | procollagen (%) |
| Comparative | 0.1 | 106.1 |
| Example 1 | 1 | 98.6 |
| | 10 | 57.8 |
| Comparative | 0.1 | 107.6 |
| Example 2 | 1 | 117.5 |
| | 10 | 113.1 |
| Control Group | — | 100 |

As can be seen in Table 2 above, the production of procollagen in Comparative Example 2 increased compared to that in the control group. Particularly, the production of procollagen in the groups treated with suitable concentrations (1-10 ppm) of the collagen in Comparative Example 2 significantly increased compared to that in the control group. However, in the case of Comparative Example 1, the effect of promoting collagen production was not observed, and in the group treated with 10 ppm of the collagen in Comparative Example 1, the production of collagen decreased rather than increased.

Accordingly, it was found that the effective concentration of the collagen peptide containing a large amount of the Gly-X-Y tripeptide having the confirmed effect needs to be determined and the synergistic effect of the collagen peptide with other candidate material needs to be considered in the preparation of the composition. For this purpose, an optimization experiment was carried out as described below.

### Experimental Example 2

### Optimization Experiment Using a Design of Experiments

In order to optimize the procollagen-producing ability of the collagen peptide component confirmed in Experimental Example 1 and other candidate materials (elastin protein, hyaluronic acid and vitamin C), a responsive surface method (RSM) was used. When the responsive surface method is used, it is possible to determine the values at which independent variables can be optimized, and it is possible to presume the functional relation between independent variables and dependent variables from data, thereby predicting what the independent variables influence response variables. Thus, it is possible to find the optimum experimental conditions between the variables. In the present invention, as the responsive surface method, a central composite design was carried out using MiniTab 14, and the results are shown in Table 3 below.

TABLE 3
Design and Its measurements (Y) by Central Composite Design

| | C1 | C2 | C3 | C4 | C5 | C6 | C7 | C8 | C9 | | |
|---|---|---|---|---|---|---|---|---|---|---|---|
| | ctp | vitC | elastin | HA | Y | StdOrder | RunOrder | [symbol] | PtType | C10 | C11 |
| 1 | 0.0 | 0.0 | 0.0 | 0.00 | 100.00 | 1 | 1 | 1 | 1 | | |
| 2 | 25.0 | 0.0 | 0.0 | 0.00 | 119.16 | 2 | 2 | 1 | 1 | | |
| 3 | 0.0 | 1.0 | 0.0 | 0.00 | 102.24 | 3 | 3 | 1 | 1 | | |
| 4 | 25.0 | 1.0 | 0.0 | 0.00 | 98.53 | 4 | 4 | 1 | 1 | | |
| 5 | 0.0 | 0.0 | 25.0 | 0.00 | 65.37 | 5 | 5 | 1 | 1 | | |
| 6 | 25.0 | 0.0 | 25.0 | 0.00 | 63.46 | 6 | 6 | 1 | 1 | | |
| 7 | 0.0 | 1.0 | 25.0 | 0.00 | 54.78 | 7 | 7 | 1 | 1 | | |
| 8 | 25.0 | 1.0 | 25.0 | 0.00 | 64.82 | 8 | 8 | 1 | 1 | | |
| 9 | 0.0 | 0.0 | 0.0 | 0.10 | 102.56 | 9 | 9 | 1 | 1 | | |
| 10 | 25.0 | 0.0 | 0.0 | 0.10 | 115.49 | 10 | 10 | 1 | 1 | | |
| 11 | 0.0 | 1.0 | 0.0 | 0.10 | 90.51 | 11 | 11 | 1 | 1 | | |
| 12 | 25.0 | 1.0 | 0.0 | 0.10 | 83.66 | 12 | 12 | 1 | 1 | | |
| 13 | 0.0 | 0.0 | 25.0 | 0.10 | 81.81 | 13 | 13 | 1 | 1 | | |
| 14 | 25.0 | 0.0 | 25.0 | 0.10 | 98.65 | 14 | 14 | 1 | 1 | | |
| 15 | 0.0 | 1.0 | 25.0 | 0.10 | 84.80 | 15 | 15 | 1 | 1 | | |
| 16 | 25.0 | 1.0 | 25.0 | 0.10 | 96.91 | 16 | 16 | 1 | 1 | | |
| 17 | 0.0 | 0.5 | 12.5 | 0.05 | 80.01 | 17 | 17 | 1 | 1 | | |
| 18 | 25.0 | 0.5 | 12.5 | 0.05 | 77.43 | 18 | 18 | 1 | 1 | | |
| 19 | 12.5 | 0.0 | 12.5 | 0.05 | 104.51 | 19 | 19 | 1 | 1 | | |
| 20 | 12.5 | 1.0 | 12.5 | 0.05 | 78.13 | 20 | 20 | 1 | 1 | | |
| 21 | 12.5 | 0.5 | 0.0 | 0.05 | 71.59 | 21 | 21 | 1 | 1 | | |
| 22 | 12.5 | 0.5 | 25.0 | 0.05 | 60.11 | 22 | 22 | 1 | 1 | | |
| 23 | 12.5 | 0.5 | 12.5 | 0.00 | 68.60 | 23 | 23 | 1 | 1 | | |
| 24 | 12.5 | 0.5 | 12.5 | 0.10 | 73.00 | 24 | 24 | 1 | 1 | | |
| 25 | 12.5 | 0.5 | 12.5 | 0.05 | 116.54 | 25 | 25 | 1 | 1 | | |
| 26 | 12.5 | 0.5 | 12.5 | 0.05 | 104.68 | 26 | 26 | 1 | 1 | | |
| 27 | 12.5 | 0.5 | 12.5 | 0.05 | 104.84 | 27 | 27 | 1 | 1 | | |
| 28 | 12.5 | 0.5 | 12.5 | 0.05 | 106.38 | 28 | 28 | 1 | 1 | | |
| 29 | 12.5 | 0.5 | 12.5 | 0.05 | 121.89 | 29 | 29 | 1 | 1 | 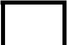 | |

*ctp: collagen peptide, vitC: vitamin C, elastin: elastin protein, and HA: hyaluronic acid.

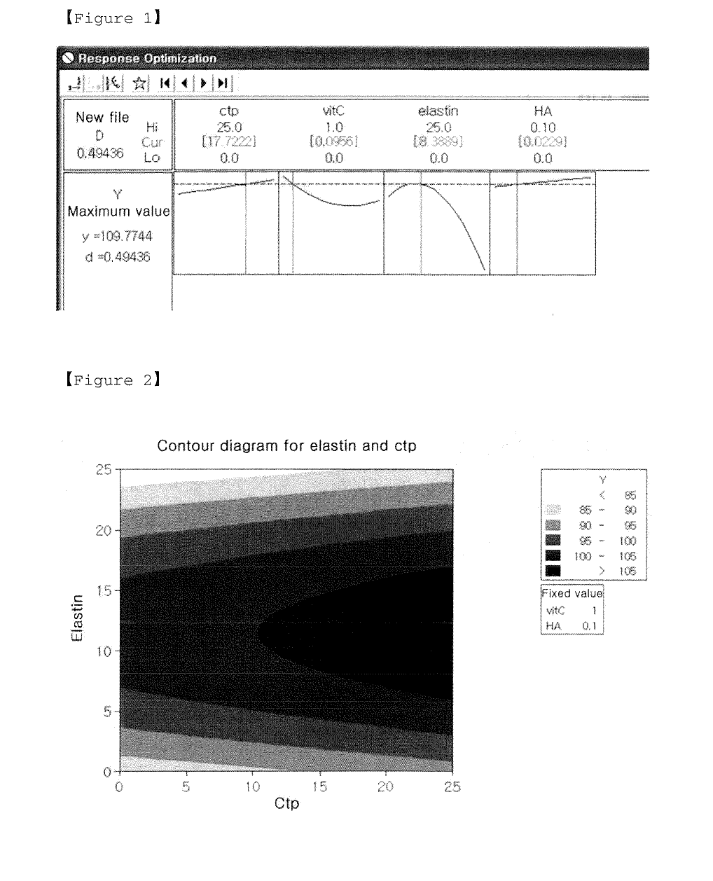

The experiment was carried out by the central composite design shown in Table 3 above, and the results of the experiment were analyzed using software. The results of the analysis are shown in FIGS. 1 and 2 and Table 4 below.

TABLE 4
Optimum concentration ratio for promoting procollagen production

| Ingredients | Concentration (ppm) | | |
|---|---|---|---|
| Collagen peptide | 25~50 | 15~25 | 0.1~15 |
| Elastin protein | 0.1~1 | 5~10 | 10~15 |
| Vitamin C | 0.2 | 0.1 | 0.1 |
| Hyaluronic acid | 0.1~1 | 1~100 | 100~500 |

As can be seen from the results of the software analysis, R² and R²(adj) were 71.8% and 60.8%, indicating that there is a relatively high degree of correlation between R² and R²(adj). Also, it can be seen that the regression model was significant. Through the response optimizer of FIG. 1, it could be seen that concentrations having the optimum conditions showing the highest values in the lager-the-better characteristics were 12.5 ppm:12.5 ppm:0.1 ppm:500 ppm. Also, through the overlaid contour plot of FIG. 2, the optimum concentration ranges can be inferred, and the results are shown in Table 4 below. The effective concentration ratio determined based on the above results is as follows:

Collagen peptide:elastin protein:vitamin C:hyaluronic acid=1:0.0001-150:0.0001-20:0.0001-50000.

### Experimental Example 3

### Experiment on Photoaging Inhibition Using Skin Replica

In order to examine the effect of the inventive composition on the inhibition of photoaging, an experiment was carried out using hairless mice as an animal model. 6-7-week-old female hairless mice (SKH, HR-1) were divided into the following three groups as shown in Table 5 below: Comparative Example 3 (normal group), Comparative Example 4 (UV control group), and Example 1, each group consisting of 8 mice. The mice were raised during the experimental period.

TABLE 5

| Classification | Treatment |
|---|---|
| Comparative | In a normal group, mice were administered |
| Example 3 | with physiological saline. |
| Comparative | As a UV control group, mice were |
| Example 4 | irradiated with UV light and administered |
| | with physiological saline. |
| Example 1 | Mice were irradiated with UV light and |
| | administered with the optimal formulation |
| | derived in Test Example 2 together with |
| | physiological saline. |

In Comparative Examples 3 and 4, mice were administered orally with 0.5 ml of physiological saline, and in Example 1, the raw materials were mixed at the optimal ratio, and 500 mg/kg bodyweight (on a solid content basis) of the mixture was added to 0.5 ml of physiological saline and administered orally to mice using a syringe. Herein, the mixture contained 666 mg/kg of the collagen peptide, 13 mg/kg of the elastin protein and 1 mg/kg of vitamin C and hyaluronic acid. The mixture was administered at the same timing for 5 days a week over a total of 5 weeks. For 2-5 weeks after the oral administration, the mice of Comparative Examples 3 and 4 and Example 1 were irradiated with UV light similar to sunlight 3 times a week. The total dose of UV light irradiated during the experimental period was 600 mJ/Cf. To objectively evaluate the effect of reducing wrinkles, a replica was sampled from the back of the hairless mice using a silicone polymer before biopsy, and to compare the degree of skin wrinkles between the groups, the skin surface was imaged using a skin visiometer, and the results are shown in FIG. 3.

As can be seen in FIG. 3, the depth or degree of skin surface wrinkles of the hairless mice of Example 1 was significantly reduced compared to that of the hairless mice of Comparative Example 4. This indicates that the composition of the present invention is effective in reducing skin wrinkles caused by UV light, thus inhibiting photoaging.

### Experimental Example 4

### Experiment on Inhibition of Photoaging Using Tissue Staining

For histopathologic observation of the hairless mice of Comparative Examples 3 and 4 and Example 1, immunohistochemical staining was performed. Skin was sampled from the back of the mice and fixed in 10% neutral formalin. Then, in order to observe the degree of expression of type 1 collagen in the skin tissue, immunohistochemical staining was performed using monoclonal IgG1 antibody.

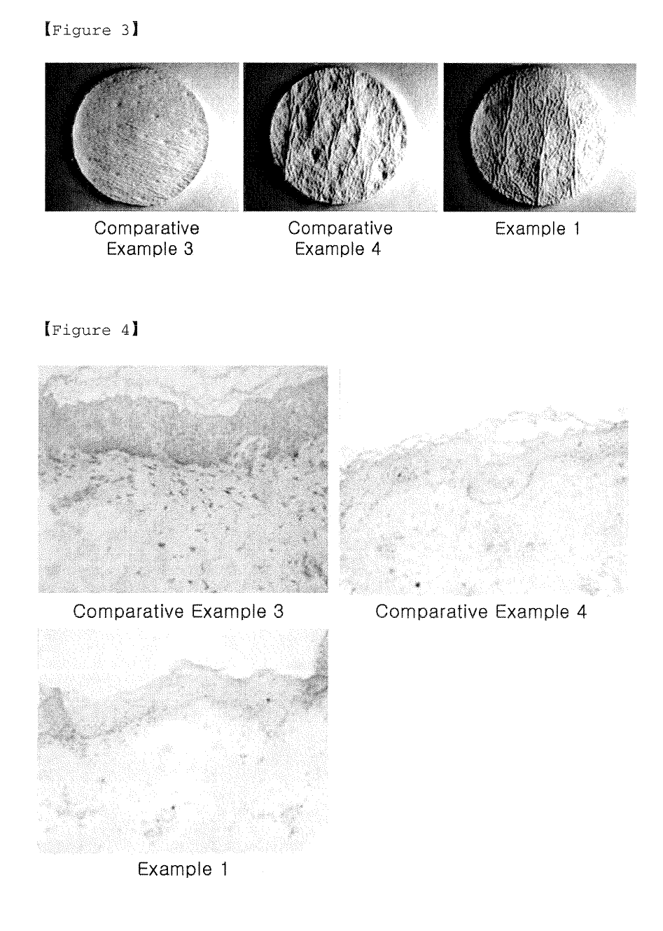

FIG. 4 shows the results of immunohistochemical staining of type 1 collagen in the skin tissue of each experimental group. As can be seen therein, the collagen of Comparative Example 4 (UV control group) was less stained than that of Comparative Example 3 (normal group), whereas the collagen at the epidermis/dermis boundary layer of the mice of Example 1 was more stained than that of Comparative Example 4. This suggests that, when the collagen peptide, the elastin protein, vitamin C and hyaluronic acid are administered in combination, the synthesis of collagen in the skin is maximized.

In addition, for the observation of general tissue status and the measurement of epidermal layer thickness, the mice of Comparative Examples 3 and 4 and Example 1 were subjected to HE (heamtoxylin & eosin) staining. To measure the thickness of the skin epidermal layer, the H&E staining slide was read at 100× magnification using a microscope, and the thicknesses of 10 randomly selected places were measured, and the measurements were averaged. The results of the measurement are shown in Table 6 below.

TABLE 6

| Classification | Thickness of the skin epidermal layer |
|---|---|
| Comparative | 5.23 ± 0.31 mm (53.5%) |
| Example 3 | |
| Comparative | 9.77 ± 0.68 mm (100%) |
| Example 4 | |
| Example 1 | 7.56 ± 0.75 mm (77.4%) |

As can be seen in Table 6 above, the thickness of the epidermal layer in Comparative Example 4 was 9.77±0.68 mm, and the epidermal thickness layer in Example 1 was 7.56±0.75 mm which was about 30% lower than that in Comparative Example 4. This indicates that the administration of the collagen peptide mixture of the present invention inhibits the phenomenon in that the skin becomes thicker due to UV irradiation.

### Experimental Example 5

### Mini-Clinical Trial

40 adult women (25-45-year old) were two groups: an experimental group and a control group. The experimental group was administered with 4 g of a pill, prepared by mixing the components shown in Table 7 below, once a day for 30 days, and the control group (placebo group) was administered with a pill, prepared by adding 1.5 g of glucose in place of the collagen peptide in Table 7 below, in the same manner as the experimental group.

After completion of the experiment, a questionnaire survey for skin conditions was performed, and the results are shown in Table 8.

TABLE 7

| | Ingredients | | | | | | | |
|---|---|---|---|---|---|---|---|---|
| | collagen | | | vitamin | hyaluronic | | | |
| | peptide | glucose | elastin | C | acid | lactose | glycerin | Xylitol |
| Experimental | 1 | — | 0.1 | 0.2 | 0.2 | 1.0 | 1 | 0.5 |
| Group | | | | | | | | |
| Control | — | 1.5 | 0.1 | 0.2 | 0.2 | 1.0 | 1 | 0.5 |
| Group | | | | | | | | |

TABLE 8

| | Experimental Group | | | | Control Group | | | |
|---|---|---|---|---|---|---|---|---|
| Survey | 20s | 30s | 40s | Total | 20s | 30s | 40s | Total |
| items | (4) | (10) | (6) | (20) | (6) | (8) | (6) | (20) |
| Moist skin | 3 | 8 | 5 | 16 (80%) | 2 | 1 | 2 | 5 (25%) |
| Elastic | 2 | 8 | 5 | 15 (75%) | 1 | 2 | 1 | 4 (20%) |
| skin | | | | | | | | |
| Reduced | 2 | 8 | 4 | 14 (70%) | 1 | 3 | 2 | 6 (30%) |
| fine | | | | | | | | |
| wrinkles | | | | | | | | |
| Good | 3 | 9 | 5 | 17 (85%) | 2 | 2 | 3 | 7 (35%) |
| makeup | | | | | | | | |
| Generally | 3 | 7 | 4 | 14 (70%) | 2 | 3 | 1 | 6 (30%) |
| improved | | | | | | | | |
| skin | | | | | | | | |
| condition | | | | | | | | |

As can be seen in Table 8 above, in the experimental group, the ratio of the subjects who felt skin moist or elastic was higher than that in the control group, and the number of the subjects, who answered that fine wrinkles were reduced and the face took makeup well, was larger than in the control group. This suggests that the use of the composition according to one embodiment of the present invention generally improves skin conditions.

### Experimental Example 6

### Experiment on In Vivo Retention Rate

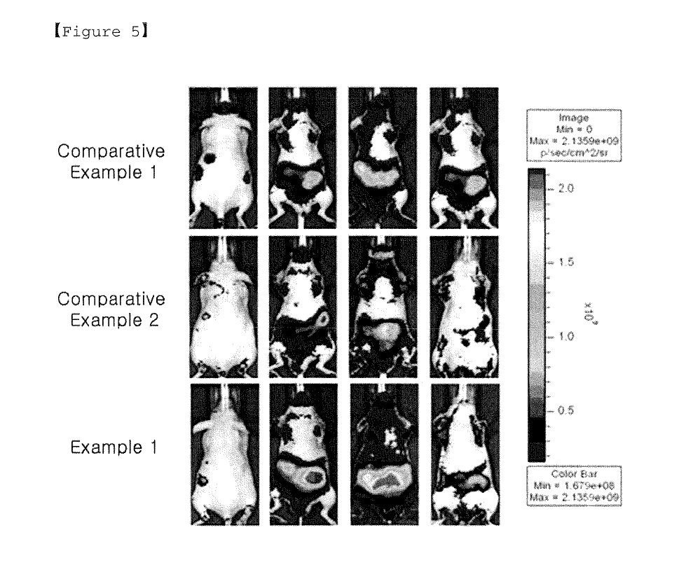

In order to compare the in vivo retention rates of the composition according to one embodiment of the present invention, general collagen and the collagen peptide alone, each of the materials of Comparative Examples 1 and 2, and Example 1 was linked with a fluorescence dye and administered orally to hairless mice. Then, the in vivo retention rate of each material with time was measured using an in vivo image analyzer, and the results of the measurement are shown in FIG. 5.

As can be seen from the results in FIG. 5, the in vivo retention rate of Example 1 that is the optimum composition was increased by 30% or more at the same time point (after 9 hours), and Example 1 showed increased in vivo retention rate even after 24 hours.

### Experimental Example 7

### Experiment on Promotion of Cell ration

In order to compare wound healing ability between the groups, a cell migration assay was performed using fibroblasts. Specifically, fibroblasts were added to a 6-well plate at a density of 1×10⁵ cells/well and allowed to adhere to each well. In this state, each of Example 1 and Comparative Examples 1 and 2 was added to the cells to a final concentration of 50 ppm. As a control group, a group not treated with any sample was prepared. After the cells have been cultured for 24 hours, the middle portion of each well was wounded with a micropipette tip, and then recovery from the wound was observed with time, and the degree of migration of the cells was evaluated based on the average gap of the wound. The results of the evaluation are shown in Table 9 below.

TABLE 9

| Classification | Average gap (%) |
|---|---|
| Control Group | 100 ± 3.4 |
| Comparative | 83 ± 4.5 |
| Example 1 | |
| Comparative | 67 ± 7.5 |
| Example 2 | |
| Example 1 | 58 ± 8.3 |

As can be seen from the results in Table 9 above, the group administered with the composition of Example showed the most excellent wound healing ability.

The composition for improving the beauty of the skin according to the present invention may be formulated in various forms as follows, but the scope of the present invention is not limited thereto.

### Formulation Example 1

### Soft Capsule

2,000 mg of the collagen peptide mixture of Example 2, 50 mg of a soybean extract, 180 mg of soybean oil, 50 mg of a red ginseng extract, 2 mg of palm oil, 8 mg of hydrogenated palm oil, 4 mg of beeswax and 6 mg of lecithin were mixed with each other, and 400 mg of the mixture was filled in each capsule, thereby preparing a soft capsule.

### Formulation Example 2

### Tablet

2,000 mg of the collagen peptide mixture of Example 2, 50 mg of a soybean extract, 100 mg of glucose, 50 mg of a red ginseng extract, 96 mg of starch and 4 mg of magnesium stearate were mixed with each other, and 40 mg of 30% ethanol was added thereto to form granules. The granules were dried at 60□ and compressed into a tablet using a tableting machine. The final weight of the content of the tablet was 400 mg.

### Formulation Example 3

### Granules

2,000 mg of the collagen peptide mixture of Example 2, 50 mg of a soybean extract, 100 mg of glucose, 50 mg of a red ginseng extract and 600 mg of starch were mixed with each other, and 100 mg of 30% ethanol was added thereto to form granules. The granules were dried at 60□ and filled in a bag. The final weight of the content of the bag was 1 g.

### Formulation Example 4

### Drink

2,000 mg of the collagen peptide mixture, 50 mg of a soybean extract, 10 g of glucose, 50 mg of a red ginseng extract, 2 g of citric acid and 188 g of purified water were mixed with each other and filled in a bottle. The final weight of the content of the bottle was 100 ml.

1. A composition for improving the beauty of the skin, which contains a collagen peptide and at least one selected from the group consisting of elastin protein, hyaluronic acid and vitamin C.

2. The composition of claim 1, wherein the collagen peptide contains a collagen tripeptide in an amount of 15-95 wt % or more based on the total weight of the collagen peptide.

3. The composition of claim 2, wherein the collagen peptide is in the form of Gly-X-Y where X and Y are selected from among the same or different amino acids.

4. The composition of claim 1, wherein the collagen peptide is contained in an amount of 1-80 wt % based on the total weight of the composition.

5. The composition of claim 1, wherein the elastin protein, hyaluronic acid and vitamin C in the composition are contained in the amounts of 1-20 wt %, 1-10 wt % and 1-20 wt %, respectively, based on the total weight of the composition.

6. The composition of claim 1, wherein the content ratio of collagen peptide:elastin protein:vitamin C:hyaluronic acid is 1:0.0001-150:0.0001-20:0.0001-50000.

7. The composition of claim 1, wherein the composition is formulated in the form of pills, tea bags, instant teas, drinks, granules, tablets or capsules.

8. A composition for promoting the production of collagen in the skin, which comprises the composition of claim 1.

9. A anti-aging composition, which comprises the composition of claim 1.

10. A composition for increasing skin moisturization, which comprises the composition of claim 1.

11. Use of a composition which contains a collagen peptide and at least one selected from the group consisting of elastin protein, hyaluronic acid and vitamin C, for improving the beauty of the skin.

12. Use of a composition which contains a collagen peptide and at least one selected from the group consisting of elastin protein, hyaluronic acid and vitamin C, for promoting the production of collagen in the skin.

13. Use of a composition which contains a collagen peptide and at least one selected from the group consisting of elastin protein, hyaluronic acid and vitamin C, for anti-aging of the skin.

14. Use of a composition which contains a collagen peptide and at least one selected from the group consisting of elastin protein, hyaluronic acid and vitamin C, for increasing skin moisturization.
